(19) United States
(12) Patent Application Publication
(10) Pub. No.: US 2014/0350538 A1
(43) Pub. Date: Nov. 27, 2014

(54) COIL-INTEGRATED PAD ASSEMBLY AND AN ELECTROMAGNETIC HYPERTHERMIA SYSTEM INCLUDING THE SAME

(71) Applicants: NATIONAL CHENG KUNG UNIVERSITY, Tainan City (TW); METAL INDUSTRIES RESEARCH & DEVELOPMENT CENTRE, Kaohsiung City (TW)

(72) Inventors: Yi-San CHANG, Tainan City (TW); Tsung-Chih YU, Tainan City (TW); Xi-Zhang LIN, Tainan City (TW); Sheng-Jye HWANG, Tainan City (TW); Ying-Jui CHAO, Tainan City (TW); Syuan-Fong CHEN, Kaohsiung City (TW); Guan-Cheng CHEN, Tainan City (TW); Tung-Chieh YANG, Linbian Township (TW); Shyang-Jye CHANG, New Taipei City (TW)

(73) Assignees: NATIONAL CHENG KUNG UNIVERSITY, Tainan City (TW); METAL INDUSTRIES RESEARCH & DEVELOPMENT CENTRE, Kaohsiung City (TW)

(21) Appl. No.: 14/274,296

(22) Filed: May 9, 2014

(30) Foreign Application Priority Data
May 24, 2013 (TW) 102118443

(51) Int. Cl. A61B 18/04

(52) U.S. Cl. CPC A61B 18/04; A61B 2018/00041
USPC 606/28

(57) ABSTRACT

A coil-integrated pad assembly for inductively heating a hyperthermia needle device includes a barrier pad and an electromagnetic unit. The barrier pad is adapted to be inserted into a patient's body and to be placed between the hyperthermia needle device and a non-target tissue of the patient's body when the hyperthermia needle device penetrates a target tissue. The electromagnetic unit has a coil that is at least partially disposed in the barrier pad and that is electrifiable to generate a magnetic field sufficient to inductively heat the hyperthermia needle device.

### CROSS-REFERENCE TO RELATED APPLICATION

This application claims priority to Taiwanese Application No. 102118443, filed on May 24, 2013.

### BACKGROUND OF THE INVENTION

1. Field of the Invention

The invention relates to a coil-integrated pad assembly, and more particularly to a coil-integrated pad assembly for inductively heating a hyperthermia needle device.

2. Description of the Related Art

In order to improve a therapeutic effect of a treatment for benign or cancerous tumor tissue, hyperthermia techniques for killing the tumor tissue, such as radio frequency ablation (RFA) and microwave ablation (MWA), have been developed in the medical field. The RFA utilizes passing radiofrequency waves through a needle-like RFA probe that is placed inside the tumor tissue in a patient's body to increase the temperature within the tumor tissue for destructing the tumor tissue. The MWA utilizes electromagnetic waves to induce oscillation of polar molecules of the tumor tissue to produce frictional heating and ultimately tissue necrosis within the tumor tissue. Although the RFA and MWA are adapted for treatments for local neoplasm, the required armamentaria are relatively expensive and unaffordable by a lot of patients.

In view of the foregoing, electromagnetic (EM) hyperthermia therapy for killing the tumor tissue has been developed. The EM hyperthermia therapy involves insertion of a magnetically inducible hyperthermia needle into the tumor tissue to be treated. Necrosis of the tumor tissue is caused by heat generated by the magnetically inducible hyperthermia needle under an induced magnetic field that is changeable at a relatively high frequency. The induced magnetic field is generated by an electromagnetic coil that is disposed outside the patient's body. The EM hyperthermia therapy gradually attracts attention since its good therapeutic effect and relatively low expense.

When performing the EM hyperthermia therapy, a conventional insulation pad is disposed beside the tumorous tissue or organ to be treated so as to prevent other tissues or organs from being injured by the magnetically inducible hyperthermia needle or the heat generated by the magnetically inducible hyperthermia needle once magnetically induced. Since the conventional insulation pad is made of a relatively rigid material, its application is limited to thoracic or abdominal surgeries where a relatively large incision is formed in the patient's body, and is not suitable for laparoscopic or endoscopic surgeries involving smaller incisions. In addition, the conventional electromagnetic coil is relatively far from the magnetically inducible hyperthermia needle inside the patient's body for being disposed outside the patient's body. In order for the magnetically inducible hyperthermia needle to generate sufficient heat to conduct the EM hyperthermia therapy, the conventional electromagnetic coil requires more electric power to induce the electromagnetic field with a sufficient strength.

### SUMMARY OF THE INVENTION

Therefore, an object of the present invention is to provide a coil-integrated pad assembly for inductively heating a hyperthermia needle device that can alleviate at least one of the aforesaid drawbacks of the prior art.

According to the present invention, a coil-integrated pad assembly for inductively heating a hyperthermia needle device comprises a barrier pad and an electromagnetic unit.

The barrier pad is adapted to be inserted into a patient's body and to be placed between the hyperthermia needle device and a non-target tissue of the patient's body when the hyperthermia needle device penetrates a target tissue.

The electromagnetic unit has a coil that is at least partially disposed in the barrier pad and that is electrifiable to generate a magnetic field sufficient to inductively heat the hyperthermia needle device.

### BRIEF DESCRIPTION OF THE DRAWINGS

Other features and advantages of the present invention will become apparent in the following detailed description of the preferred embodiment with reference to the accompanying drawings, of which:

FIG. 1 is a schematic fragmentary perspective view of a coil-integrated pad assembly for inductively heating a hyperthermia needle device according to the preferred embodiment of the present invention;

FIG. 2 is a fragmentary partly-sectional view of the coil-integrated pad assembly;

FIG. 3 is a schematic perspective view, illustrating the coil-integrated pad assembly being wound to be inserted into a patient's body through a relatively small incision made in a patient's body; and

FIG. 4 is a schematic partly-sectional view, illustrating an electromagnetic hyperthermia system according to the preferred embodiment of this invention, including the coil-integrated pad assembly and a hyperthermia needle device.

### DETAILED DESCRIPTION OF THE PREFERRED EMBODIMENTS

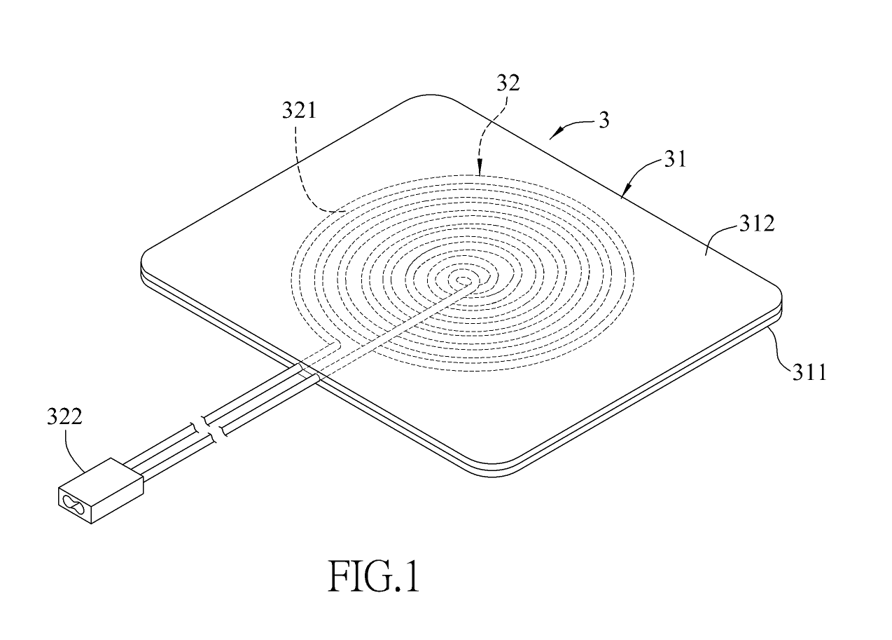

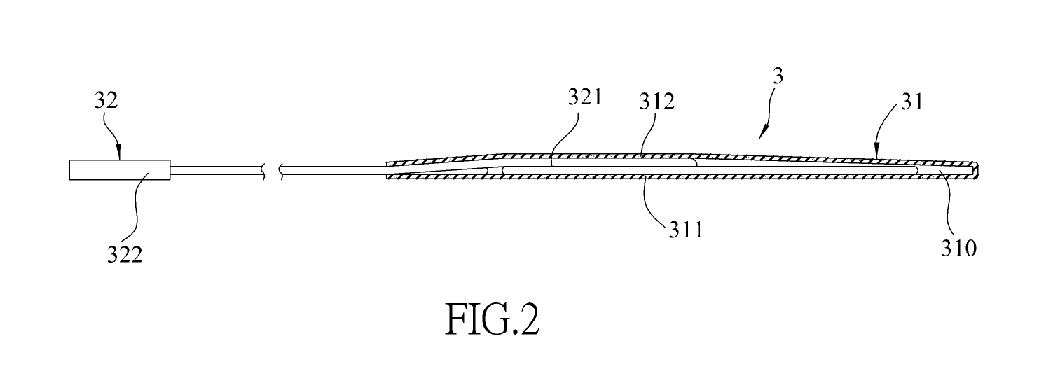

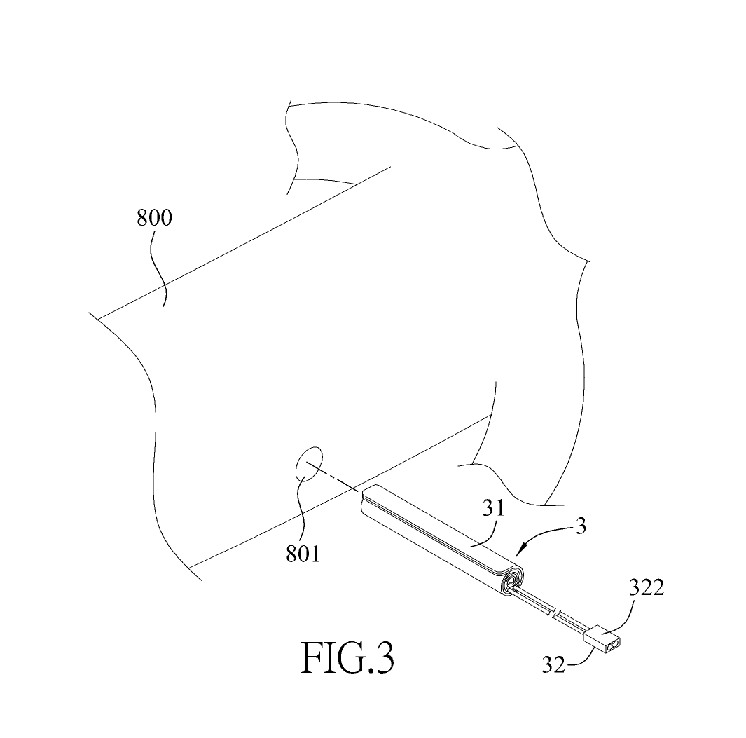

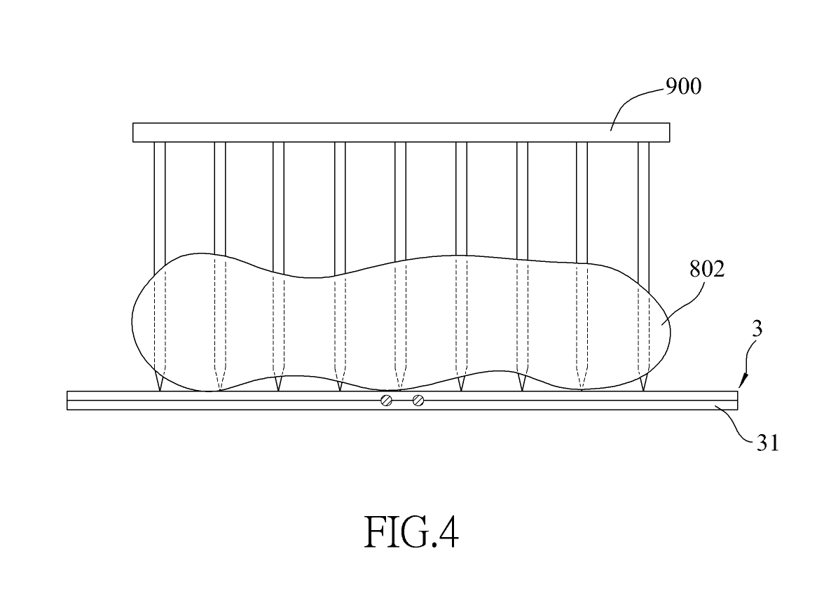

Referring to FIGS. 1 to 3, a coil-integrated pad assembly 3 for inductively heating a hyperthermia needle device according to the preferred embodiment of the present invention is shown to include a barrier pad 31 and an electromagnetic unit 32. Referring further to FIG. 4, the coil-integrated pad assembly 3 is assembled with a hyperthermia needle device 900 to constitute an electromagnetic hyperthermia system according to the preferred embodiment of this invention. The hyperthermia needle device 900 is adapted to penetrate a target tissue 802 inside a patient's body 800 during an electromagnetic (EM) hyperthermia therapy.

The barrier pad 31 is adapted to be inserted into the patient's body 800 and to be placed between the hyperthermia needle device 900 and a non-target tissue or organ (not shown) of the patient's body 800 when the hyperthermia needle device 900 penetrates a target tissue 802.

The electromagnetic unit 32 has a coil 321 that is at least partially disposed inside the barrier pad 31 in a flexible fashion. The coil 321 is electrifiable to generate a high frequency magnetic field to inductively heat the hyperthermia needle device 900. When the temperature of the target tissue 802 is increased due to the heat generated by the hyperthermia needle device 900, the barrier pad 31 is able to prevent the non-target tissue or organ from being injured by the heat generated by the hyperthermia needle device 900.

In this preferred embodiment, the barrier pad 31 has a base plate portion 311 and a top plate portion 312 that is disposed over and connected to the base plate portion 311. The top and base plate portions 312, 311 cooperatively define an installation space 310. In addition, the barrier pad 31 obstructs the hyperthermia needle device 900 from puncturing the non-target tissue during insertion of the hyperthermia needle device 900 into the target tissue 802. Preferably, the barrier pad 31 is made of a flexible material selected from rubbers, such as silicone rubbers and fabrics.

The coil 321 is at least partially installed in the installation space 310 and includes two free ends and a central portion connected between the two free ends. The central portion is wound in a spiral manner and the two free ends extend outwardly from the installation space 310. Preferably, the electromagnetic unit 32 further has a connector 322 that is disposed outside the installation space 310, that is connected to the free ends of the coil 321 and that is electrically coupled to a high frequency AC power source (not shown) and transmits a high frequency AC power from the high frequency AC power source to the coil 321, such that the coil 321 generates a high frequency magnetic field. In this preferred embodiment, the coil 321 is designed to have a relatively small diameter and is made of a flexible metal material so as to be deformable together with the barrier pad 31. Since the feature of this invention does not reside in the shape of the coil 321, which may be readily appreciated by those skilled in the art, details of the same are omitted herein for the sake of brevity.

When the coil-integrated pad assembly 3 is used in a surgery, such as laparoscopic or endoscopic surgery, involving formation of a relatively small incision 801 in the skin of the patient's body 800, the barrier pad 31 is curled in a scroll manner and before being inserted into the patient's body 800 through the incision 801. After the barrier pad 31 is placed and uncurled in a predetermined position within the patient's body 800, the connector 32 is left outside the patient's body 800 and is electrically coupled to the high frequency AC power source. After the hyperthermia needle device 900 penetrates the target tissue 802 (see FIG. 4), the high frequency AC power source is powered on and thus enables the coil 321 to generate the high frequency magnetic field. The temperature of the target tissue 802 is increased by the heat generated by the hyperthermia needle device 900 under the high frequency magnetic field.

During the electromagnetic hyperthermia therapy, the barrier pad 31 is able to obstruct the insertion movement of the hyperthermia needle device 900 so as to avoid penetration of the hyperthermia needle device 900 into the non-target tissue or organ and to prevent heat conduction from the hyperthermia needle device 900 to the non-target tissue or organ. Besides, since the barrier pad 31 is made of a flexible material, the barrier pad 31 is deformable to be contoured in accordance with surgery requirements. Moreover, the coil 321 disposed in the barrier pad 31 is arranged to be proximate to the hyperthermia needle device 900 in the patient's body 800, and thus the high frequency magnetic field generated by the coil 321 is consumed by heat generation of the hyperthermia needle device 900 in a highly efficient and economic manner.

Alternatively, when the coil-integrated pad assembly 3 of this invention is to be applied to a surgery involving formation of a relatively large incision in the patient's body 800, the barrier pad 31 and the coil 321 may be designed to be inflexible. In such case, by virtue of the proximate arrangement of the hyperthermia needle device 900 and the coil 321, the high frequency magnetic field generated by the coil 321 is likewise consumed by the heat generation of the hyperthermia needle device 900 in a highly efficient and economic manner.

While the present invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

What is claimed is:

1. A coil-integrated pad assembly for inductively heating a hyperthermia needle device, comprising:
a barrier pad that is adapted to be inserted into a patient's body and to be placed between the hyperthermia needle device and a non-target tissue of the patient's body when the hyperthermia needle device penetrates a target tissue; and
an electromagnetic unit having a coil that is at least partially disposed in said barrier pad and that is electrifiable to generate a magnetic field sufficient to inductively heat the hyperthermia needle device.

2. The coil-integrated pad assembly as claimed in claim 1, wherein said barrier pad has a base plate portion and a top plate portion that is disposed over and connected to said base plate portion, said top and base plate portions cooperatively defining an installation space, said coil being at least partially installed in said installation space.

3. The coil-integrated pad assembly as claimed in claim 2, wherein said electromagnetic unit further has a connector connected to said coil and disposed outside said installation space.

4. The coil-integrated pad assembly as claimed in claim 1, wherein said barrier pad is made of a flexible material, said electromagnetic unit being at least partially disposed in said barrier pad in a flexible fashion.

5. An electromagnetic hyperthermia system, comprising:
a hyperthermia needle device adapted to penetrate a target tissue inside a patient's body; and
a coil-integrated pad assembly including
a barrier pad that is adapted to be inserted into the patient's body and to be placed between said hyperthermia needle device and a non-target tissue of the patient's body, and
an electromagnetic unit that has a coil which is at least partially disposed inside said barrier pad, and which is electrifiable to generate a high frequency magnetic field to inductively heat said hyperthermia needle device.

6. The electromagnetic hyperthermia system as claimed in claim 5, wherein said barrier pad has a base plate portion and a top plate portion that is disposed over and connected to said base plate portion, said top and base plate portions cooperatively defining an installation space, said coil being at least partially installed in said installation space.

7. The electromagnetic hyperthermia system as claimed in claim 6, wherein said electromagnetic unit further has a connector connected to said coil and disposed outside said installation space.

8. The electromagnetic hyperthermia system as claimed claim 5, wherein said barrier pad is made of a flexible material, said electromagnetic heating unit being at least partially disposed in said barrier pad in a flexible fashion.
